(19) United States
(12) Patent Application Publication
(10) Pub. No.: US 2004/0247560 A1
(43) Pub. Date: Dec. 9, 2004

(54) Pharmaceutical preparations comprising iox exchange resins charged with active ingrdients

(76) Inventors: Martin Dirk, Langenfeld (DE); Wolfgang Block, Leverkusen (DE); Hans-Jurgen Hamann, Dormagen (DE)

Correspondence Address: Richard S Bullitt, Bayer HealthCare, 36 Columbia Road, PO Box 1910, Morristown, NJ, 07962-1910, US

(21) Appl. No.: 10/483,635

(22) PCT Filed: Jul. 4, 2002
(86) PCT No.: PCT/EP02/07417

(30) Foreign Application Priority Data
Jul. 17, 2001 (DE) 101 34 719.7

(51) Int. Cl. A61K 31/785

(52) U.S. Cl. 424/78.15

(57) ABSTRACT

The present invention relates to pharmaceutical preparations comprising one or more active substances bound to an ion exchanger. In order to improve palatability and increase stability at least 90% of these active substance/ion exchanger particles are smaller than 50 μm.

The present invention relates to pharmaceutical preparations comprising one or more active substances bound to an ion exchanger. In order to improve palatability and increase stability at least 90% of these active substance/ion exchanger particles are smaller than 50 μm.

It has long been known to bind active pharmaceutical substances to ion exchange resins in order to make it easier to administer, for example, active substances with a pronounced inherent odour (CH 383 552). It is also known to bind active pharmaceutical substances to ion exchange resins in order to effect uniform release of the active substance over a prolonged period (DE 30 28 082). It is additionally known to bind active anthelmintic substances to ion exchange resins in order to influence the taste of the active substances (DE 30 28 082). By binding to ion exchange resins it is also possible to mask the bitter taste of quinolone antibiotics, so permitting administration to animals (EP-A-295 495).

The abovementioned text describes formulations of bitter-tasting quinolone carboxylic acid derivatives which are bound to ion exchange resins, and their production. The ion exchange resins in question include weakly acidic cationic types, whose matrix may be gel-like or macroporous. Suitable base monomers for the ion exchangers are polymerizable monomers which by means of appropriate side chains may be functionalized to give cation exchange resins. The ion exchangers are known under the trade names Lewatit®, Amberlite®, Purolite® or Dowex®. Corresponding formulations have been described in veterinary medicine as feed medicaments for pigs.

However, formulations containing the loaded cation exchange resins produced in accordance with EP-A-295 495 still have disadvantages when administered orally to domestic animals, especially cats, in terms of acceptance, for example. These disadvantages are attributed to a sandy taste sensation as a result of ion exchanger particles larger than about 50 μm.

U.S. Pat. No. 3,138,525 describes improving the palatability by grinding a strongly acidic ion exchanger loaded with amprotropine. However, the size reduction to a particle size of 40-250 μm described therein is not sufficient to improve acceptance in the case of cats. WO 89/12 452 describes the grinding of cholestyramine, a strongly basic ion exchanger, using a cutting mill. 75% of the resultant particles are smaller than 65 μm and 30% are smaller than 30 μm. Cutting mills are suitable for grinding soft polymers, such as cholestyramine, but not for more friable polymers such as the cation exchange resins described above. The use of attrition mills has likewise been described only for the comminution of cholestyramine (EP 26 574).

It has now been found that acidic ion exchange resins as well, and also their formulations, can be ground by appropriate grinding processes in such a way that at least 90% of the particles have a size of less than 50 μm. Preparations which comprise these ground ion exchange resins possess unexpectedly good technical and pharmaceutical properties and, in addition, are readily accepted by domestic animals, for example, especially cats.

The invention accordingly provides:

A pharmaceutical preparation comprising one or more active substances bound to a cation exchanger, characterized in that the loaded cation exchanger is dispersed in a carrier medium and at least 90% of the dispersed ion exchanger particles have a size of less than 50 μm.

A process for producing pharmaceutical preparations according to claim 1, in which

(a) either the ion exchanger loaded with active substance, together if desired with further auxiliaries, is dispersed in a carrier and ground to the desired particle size,

(b) or the ion exchanger alone is first ground to the desired particle size and loaded with active substance and then the loaded ion exchanger is converted into the final formulation.

The use of cation exchangers loaded with active substances, at least 90% of the exchanger particles having a size of less than 50 μm, for producing pharmaceutical preparations for oral administration.

The cationic ion exchange resins may have, for example, a gel-like or macroporous matrix. Suitable base monomers for the ion exchangers include polymerizable monomers which by means of appropriate functionalization can be converted into cation exchange resins. Examples of suitable monomers include (meth)acrylates, (meth)acrylonitrile and styrene derivatives. Further comonomers used to prepare the base polymers include polyvinyl compounds such as divinylbenzene, ethylene glycol dimethacrylate or methylenebisacrylamide, for example. Condensation resins which lead to cationic exchangers are also suitable, examples being phenol-formaldehyde resins with appropriate functional groups.

The ion exchangers which can be used are not novel. Further details of various ion exchanger types and their preparation can be found, for example, in Ullmann's Encyclopedia of Industrial Chemistry (Release 2001, 6th Edition). The preferred macroporous resins may have different pore volumes. The degree of crosslinking of the suitable ion exchange resins should preferably be up to 20% and with particular preference up to 12%. Prior to grinding, the synthetic resins usually have particle sizes of from 1 to 300 μm, preferably from 10 to 200 μm. Examples of customary commercial ion exchange resins are Lewatit®, Amberlite®, Dowex® and Purolite®.

Strongly acidic ion exchangers used are preferably those based on poly(styrene, divinylbenzene)sulphonic acid. Examples that may be mentioned include:

Amberlite IRP 69: poly(styrene, divinylbenzene)sulphonic acid in Na form, particle size (before grinding): 10-25%>75 μm, max. 1%>150 μm, K exchange capacity: 110-135 mg/g corresponding to 2.75-3.38 eq/kg

Purolite C 100H MR: poly(styrene, divinylbenzene) sulphonic acid in H form, particle size (before grinding): max. 1%>150 μm, exchange capacity: at least 3.2 eq/kg

Purolite C 100 MR: poly(styrene, divinylbenzene) sulphonic acid in Na form, corresponds to Amberlite IRP 69

Lewatit Catalyst K 1481: poly(styrene, divinylbenzene) sulphonic acid in H form, particle size (before grinding): min. 97%<30 μm, exchange capacity: 5.0 eq/kg

Lewasorb SW 12: poly(styrene, divinylbenzene) sulphonic acid in Na form, corresponds otherwise to Lewatit K 1481.

As ion exchangers it is preferred to use weakly acidic cation exchangers, especially those based on methacrylic acid-divinylbenzene copolymers. Examples include:

Amberlite IRP 64: methacrylic acid-divinylbenzene copolymer in H form, particle size (before grinding): 15-30%>75 μm, max. 1%>150 μm, exchange capacity: min. 10 eq/kg

Purolite C 115 K MR: methacrylic acid-divinylbenzene copolymer in potassium form, particle size (before grinding): max. 1%>150 μm

Purolite C 115H MR: methacrylic acid-divinylbenzene copolymer in H form, otherwise like Purolite C 115 K MR.

Lewatit CNP 105: macroporous methacrylic acid-divinylbenzene copolymer in H form, exchange capacity min. 1.4 eq/1.

Active pharmaceutical substances having a basic function which are capable of binding to cation exchangers can be used. This is appropriate in particular in the case of medicaments with an unpleasant odour or which lead to an unpleasant taste sensation when administered orally. Examples that may be mentioned of active substances of this kind include, quinolone antibiotics, as disclosed inter alia in the following documents: U.S. Pat. No. 4,670,444 (Bayer AG), U.S. Pat. No. 4,472,405 (Riker Labs), U.S. Pat. No. 4,730,000 (Abbott), U.S. Pat. No. 4,861,779 (Pfizer), U.S. Pat. No. 4,382,892 (Daiichi), U.S. Pat. No. 4,704,459 (Toyama); specific examples include the following: ciprofloxacin, enrofloxacin, ibafloxacin, sarafloxacin, difloxacin, binfloxacin, danofloxacin, marbofloxacin, benofloxacin, ofloxacin, orbifloxacin, tosufloxacin, temafloxacin, pipemidic acid, norfloxacin, pefloxacin, ofloxacin, fleroxacin. Further suitable quinolone antibiotics include the compounds described in WO 97/31001, especially 8-cyano-1-cyclopropyl-7-((1S,6S)-2,8-diazabicyclo[4.3.0]nonan-8-yl)-6-fluoro-1,4-dihydro-4-oxo-3-quinolinecarboxylic acid of the formula

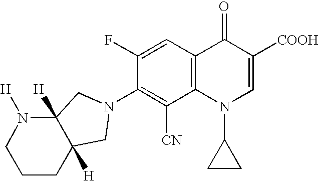

Also suitable, furthermore, are other active substances having a suitable basic function, as specified inter alia in the following documents: U.S. Pat. No. 3,536,713, U.S. Pat. No. 3,714,159, U.S. Pat. No. 3,682,930, U.S. Pat. No. 3,177,252; specific examples include the following active substances: ampicillin, amoxicillin, cephazolin, cefotiam, ceftizoxim, cefotaxim, cefodizim, ceftriaxon, ceftazidim, cefsulodin, cefalexin, cefaclor, cefadroxil, cefpodoximproxetil, cefetametpivoxil, cefixim, ceftibuten, loracarbef, imipenem, aztreonam, streptomycin, neomycin, kanamycin, spectinomycin, tetracycline, oxytetracycline, doxycycline, minocycline, erythromycin, clarithromycin, roxithromycin, azithromycin, spiramycin, sulfadiazin, sulfamethoxazole, sulfalene, sulfadoxin, trimethoprim, tetroxoprim, metronidazole, nimorazole, timidazole, lincomycin, clindamycin, vancomycin, teicoplanin, isoniazid, pyrazinamide, ethambutol, rifampicin, clotrimazole, econazole, isoconazole, oxiconazole, bifonazole, tioconazole, fenticonazole, miconazole, ketoconazole, itraconazole, fluconazole, terbinafin, naftifin, amorolfin, flucytosin, amphotericin B, nystatin, quloroquin, mefloquin, quinine, primaquin, halofantrin, proguanil, pyrimethamine, melarsoprol, nifurtimox, pentamidine, amantadine, tromantadine, aciclovir, ganciclovir, vidarabine, didanosine, zalcitabin, pyrantel, mebendazole, albendazole, tiabendazole, diethylcarbamazine, pyrvinium, oxamniquin, ambroxol, loperamide, ketotifen, metoclopramid, flupirtin.

The ion exchange resins loaded with active substance are prepared in water or polar organic solvents, such as alcohols, for example, such as methanol or ethanol, ketones, such as acetone, or mixtures thereof. Water is particularly preferred. Ion exchanger and active substance are stirred in the medium at room temperature or elevated temperature until the active substance is fully bound. Subsequently, the dispersing medium is separated off by filtration, centrifugation or decanting and the residue is dried.

Grinding of the ion exchanger may take place in the dry state or, preferably, by wet grinding, with examples including air jet grinding and grinding with a bead mill. The ion exchanger can be ground unloaded, although grinding in the loaded state is preferred. For this purpose the ion exchanger, together where appropriate with further auxiliaries, such as wetting agents, preservatives or viscosity-increasing substances, is dispersed in a suitable carrier and ground. Suitable carrier liquids include water, organic solvents, fatty mineral oils or mixtures thereof, but especially fatty oils.

The ion exchangers used in accordance with the invention are ground so that 90% of the particles are smaller than 50 μm [D(0.9)=50 μm], preferably smaller than 20 μm [D(0.9)=20 μm], with particular preference smaller than 10 μm [D(0.9)=10 μm]. In one preferred embodiment the particles are always smaller than 100 μm, preferably 75 μm. The particle sizes indicated here are always as determined by laser scattered-light measurement (e.g. using a Malvern Mastersizer or the like).

Surprisingly it has been found that the pharmaceutical preparation of the invention is distinguished, owing to the low particle size of the loaded ion exchanger [D(0.9)<50 μm], by excellent palatability, for cats, for example.

In liquid formulations, furthermore, the grinding of the ion exchanger not only markedly delays the formation of a sediment but also means that the sediment is very easy to reagitate. The better reagitatability of the finely ground ion exchanger, in particular, was surprising, since finely particulate, sedimenting suspensions normally tend toward poor redispersibility (caking) owing to the high specific surface area and hence the high adhesion forces of the particles.

The pharmaceutical preparations of the invention are suitable in general for application in both humans and animals. They are preferably encountered in animal keeping and animal breeding in connection with livestock, breeding stock, zoo animals, laboratory animals, experimental animals and pets.

The livestock and breeding stock include mammals such as, for example, cattle, horses, sheep, pigs, goats, camels, water buffalo, donkeys, rabbits, fallow deer, reindeer, fur-bearing animals such as, for example, mink, chinchilla, racoon, and birds such as, for example, chickens, geese, turkeys, ducks, pigeons and bird species kept in homes and in zoos.

Laboratory and experimental animals include mice, rats, guinea pigs, golden hamsters, dogs and cats.

Pets include rabbits, hamsters, guinea pigs, mice, horses, reptiles, appropriate bird species, dogs and cats.

Fish may also be mentioned, including farmed fish, breeding fish, aquarium fish and ornamental fish of all age levels which live in fresh water and salt water. The farmed fish and breeding fish include, for example, carp, eel, trout, whitefish, salmon, bream, roach, rudd, chub, sole, plaice, halibut, Japanese yellowtail (Seriola quinqueradiata), Japanese eel (Anguilla japonica), red seabream (Pagurus major), seabass (Dicentrarchus labrax), grey mullet (Mugilus cephalus), pompano, gilthead seabream (Sparus auratus), Tilapia spp., cichlid species such as, for example, plagioscion, channel catfish. The compositions of the invention are especially suitable for treating fish fry, e.g. carp of 2 to 4 cm in body length. The compositions are also very suitable in eel feeding.

The preparations of the invention are used preferably in connection with pets such as hamsters, rabbits, guinea pigs, cats and dogs. They are particularly suitable for use with cats.

Administration may be carried out either prophylactically or therapeutically.

The preparations of the invention are preferably administered orally.

Medicament preparations suitable for animals are those, for example, where a part is played by the improvement of taste during consumption or where the aim is for retarded release of active substance following administration.

Examples of such preparations include those such as powders, premixes or concentrates, granules, pellets, tablets, boluses, capsules, or liquid or semi-solid medicament forms such as suspensions or pastes, which are administered orally, for example. The latter are prepared by suspending the resin, loaded with active substance, in a carrier liquid, where appropriate with the addition of further auxiliaries such as wetting agents, colorants, absorption promoters, preservatives, antioxidants or light stabilizers.

By adding substances which raise the viscosity these suspensions may also be administered in the form of what are known as semisolid preparations such as ointments, for example. Formulations of this kind can be used in particular as an oral paste in cats, dogs and horses.

In order to produce solid preparations, the resin, loaded with active substance, is mixed with suitable excipients, where appropriate with the addition of auxiliaries, and is brought into the desired form.

Excipients that may be mentioned include all physiologically compatible inert substances. They include both organic and inorganic substances. Examples of inorganic substances are sodium chloride, carbonates such as calcium carbonate, hydrogen carbonates, aluminium oxides, silicas, aluminas, precipitated or colloidal silica, and phosphates.

Organic substances are, for example, sugar, cellulose, foodstuffs and feedstuffs such as milk powder, animal meals, cereal meals and cereal shreds, and starches.

Auxiliaries are preservatives, antioxidants and colorants, which have already been set out above.

Further suitable auxiliaries are lubricants and glidants, such as, for example, magnesium stearate, stearic acid, talc, bentonites, disintegration promoters such as starch or crosslinked polyvinylpyrrolidone, binders such as, for example, starch, gelatine or linear polyvinylpyrrolidone, and dry binders such as microcrystalline cellulose.

For preparing suspensions the resins loaded with active substance are dispersed as homogeneously as possible in a liquid carrier medium, where appropriate with the assistance of other auxiliaries such as wetting agents, preservatives or viscosity enhancers.

Carrier liquids that may be mentioned include all homogeneous solvents and solvent mixtures, but especially water, liquid paraffin and fatty oils, e.g. neutral oil.

In the preparations according to the present invention the ion exchange resin loaded with active substance is preferably present in an amount of 1 to 50% (w/w), more preferably in an amount of 5 to 25% (w/w) based on the total weight of the preparation.

The carrier liquid is used in an amount suitable to adjust the required consistency, usually 50 to 99% (w/w), preferably 70 to 95% (w/w) based on the total weight of the preparation are used.

Wetting agents (dispersants) include:

anionic surfactants including emulsifiers such as Na lauryl sulphate, fatty alcohol ether sulphates, mono/dialkyl polyglycol ether orthophbsphoric ester monoethanolamine salt, ligninsulphonates or dioctyl sulphosuccinate

cationic surfactants, including emulsifiers such as cetyltrimethylammonium chloride

ampholytic surfactants, including emulsifiers such as di-Na N-lauryl-β-iminodipropionate or lecithin

nonionic surfactants, including emulsifiers, such as polyoxyethylated castor oil, polyoxyethylated sorbitan monooleate, sorbitan monostearate, glycerol monostearate, polyoxyethylene stearate, alkylphenol polyglycol ethers, polyethylene-polypropylene block copolymers.

The nonionic surfactants are particularly preferred.

Wetting agents are preferably used in an amount of 0.1 to 10% (w/w), more preferably 0.5 to 5% (w/w) based on the total weight of the preparation.

Examples of further auxiliaries include:

Colorants, i.e., all colorants which are approved for use in humans or animals, and which may be in dissolved or suspended form. Colorants are preferably used in an amount of 0.001 to 5% (w/w), more preferably 0.01 to 2% (w/w) based on the total weight of the preparation.

Antioxidants such as, for example, sulphites or metabisulphites such as potassium metabisulphite, ascorbic acid, butylated hydroxytoluene, butylated hydroxyanisole, tocopherols. Antioxidants are preferably used in an amount of 0.001 to 5% (w/w), more preferably 0.01 to 2% (w/w) based on the total weight of the preparation.

Thickeners and viscosity enhancers such as, for example, inorganic thickeners such as bentonites, colloidal silica, aluminium monostearate, organic thickeners such as cellulose derivatives, polyvinyl alcohols and their copolymers, acrylates and methacrylates, alginates, gelatin, poly-vinylpyrrolidone, polyethylene glycols, waxes, gum arabic and xanthan gum, or mixtures of the substances listed. Colorants are preferably used in an amount of 0.01 to 10% (w/w), more preferably 0.01 to 5% (w/w) based on the total weight of the preparation.

With preference, the preparations of the invention are liquid or semi-liquid suspensions.

By adding substances which increase the viscosity it is also possible to administer these suspensions as semi-solid preparations such as pastes, for example. In particular, formulations of this kind can be used as oral pastes in animals.

Furthermore, the ion exchange resins loaded with active substance may be added, per se or in the form of premixes or feed concentrates, to the feed.

Premixes and feed concentrates are mixtures of the active substance with a suitable carrier substance.

Carrier substances include the feed ingredients or mixtures thereof, and also the inert carrier substances referred to earlier on above.

They may additionally comprise further auxiliaries, such as substances which regulate the fluidity and miscibility, such as silicas, bentonites, and ligninsulphonates, for example. They may also include added antioxidants such as BHT or preservatives such as sorbic acid or calcium propionate. Additionally, liquids such as liquid paraffins, plant oils and propylene glycols may be admixed to the premixes for the purpose of binding dust.

The resins loaded with active substance may be present alone in the formulations or in a mixture with other active substances, mineral salts, trace elements, vitamins, proteins, colorants, fats or flavourings.

The ion exchange resins loaded with active substance may be administered together with the feed.

The feed includes feed ingredients of plant origin such as hay, beets, cereals, cereal by-products, feed ingredients of animal origin such as meat, fats, dairy products, bone meal, fish products, and also feed ingredients such as vitamins, proteins, amino acids, for example DL-methionine, salts such as calcium carbonate and sodium chloride. The feed also includes supplementary, formulated and compounded feedstuffs. These contain feed ingredients in a composition which ensures a balanced diet in terms of energy and protein supply and the supply of vitamins, mineral salts and trace elements.

### PREPARATION EXAMPLES

### Example 1

3.86 kg of enrofloxacin and 19.24 kg of Amberlite IRP 64 are suspended in 76.90 kg of purified water and the suspension is stirred at room temperature for at least 8 hours. It is then transferred to a filter dryer, filtered and dried at 85° C. 17.96 kg of the resulting loaded ion exchanger are suspended together with 60 g of colloidal silica (e.g. Aerosil 200) in 100.40 kg of neutral oil (e.g. Miglyol 812) and the suspension is ground with a bead mill (e.g. DynoMill KD 6, W.A. Bachofen AG). At least 90% of the resulting particles are smaller than 10 μm.

### Example 2

5.00 kg of 8-cyano-1-cyclopropyl-7-((1S,6S)-2,8-diazabicyclo[4.3.0]nonan-8-yl)-6-fluoro-1,4-dihydro-4-oxo-3-quinoline-carboxylic acid and 20.00 kg of Purolite C 100H MR are suspended in 80.00 kg of purified water and the suspension is stirred at room temperature for at least 8 hours. After sedimentation, the supernatant is run off. The residue is dried with a paddle dryer at 75° C. 24.00 kg of the resulting loaded ion exchanger are suspended together with 2.40 kg of aluminium stearate in 213.60 kg of thinly liquid paraffin and the suspension is ground with a bead mill (e.g. Drais PM 25 TEX). At least 90% of the suspended particles are thereafter smaller than 20 μm.

### Example 3

3.50 kg of flupirtin and 5.25 kg of Amberlite IRP 69 are suspended in 40.00 kg of 50% ethanol (v/v) and the suspension is stirred at 40° C. for at least 12 hours. It is then transferred to a filter dryer, filtered and dried at 60° C. 8.50 kg of the loaded ion exchanger are ground with a spiral jet mill (e.g. Alpine 100 AS) so that at least 90% of the resulting particles are smaller than 50 μm. The ground ion exchanger and 0.68 kg of methylcellulose are subsequently dispersed in 30.60 kg of purified water.

### Example 4

5.00 kg of 8-cyano-1-cyclopropyl-7-((1S,6S)-2,8-diazabicyclo[4.3.0]nonan-8-yl)-6-fluoro-1,4-dihydro-4-oxo-3-quinoline-carboxylic acid and 20.00 kg of Amberlite IRP 64 are suspended in 75.00 kg of purified water and the suspension is ground with a bead mill (e.g. DynoMill KD 6, W.A. Bachofen AG). Then 2 kg of highly viscous hydroxyethylcellulose (e.g. Natrosol 250 HX) are incorporated with vigorous stirring to form a paste. At least 90% of the suspended particles are smaller than 20 μm.

### Example 5

3.0 kg of 8-cyano-1-cyclopropyl-7-((1S,6S)-2,8-diazabicyclo[4.3.0]nonan-8-yl)-6-fluoro-1,4-dihydro-4-oxo-3-quinoline-carboxylic acid and 17.0 kg of Amberlite IRP 64 are suspended in 80 kg of purified water and the suspension is stirred at room temperature for at least 8 hours. It is then transferred to a filter dryer, filtered and dried at 85° C. 10.0 kg of the resulting loaded ion exchanger are suspended in 83.0 kg of neutral oil (e.g. Miglyol 812) and the suspension is ground with a bead mill (e.g. DynoMill KD 6, W.A. Bachofen AG). At least 90% of the resulting particles are smaller than 10 μm. The ground suspension is subsequently converted into a paste, in a homogenizer, by adding 7.0 kg of Aerosil 200.

1. Pharmaceutical preparation comprising one or more active substances bound to a cation exchanger, characterized in that the loaded cation exchanger is dispersed in a carrier medium and at least 90% of the dispersed ion exchanger particles have a size of less than 50 μm.

2. Pharmaceutical preparation according to claim 1, characterized in that at least 90% of the dispersed ion exchanger particles have a size of less than 20 μm.

3. Pharmaceutical preparation according to claim 1, characterized in that the ion exchanger is a weakly acidic ion exchanger.

4. Pharmaceutical preparation according to claim 1, in the form of a suspension.

5. Pharmaceutical preparation according to claim 1, in the form of a paste.

6. Pharmaceutical preparation according to claim 1, characterized in that the active substance bound to the ion exchanger is a quinolone antibiotic.

7. Pharmaceutical preparation according to claim 1, characterized in that the active substance bound to the ion exchanger is enrofloxacin.

8. Pharmaceutical preparation according to claim 1, characterized in that the active substance bound to the ion exchanger is an 8-cyano-1-cyclopropyl-7-((1S,6S)-2,8-diazabicyclo[4.3.0]nonan-8-yl)-6-fluoro-1,4-dihydro-4-oxo-3-quinolinecarboxylic acid.

9. Process for producing pharmaceutical preparations according to claim 1, in which
(a) either the ion exchanger loaded with active substance, together if desired with further auxiliaries, is dispersed in a carrier and ground to the desired particle size,
(b) or the ion exchanger alone is first ground to the desired particle size and loaded with active substance and then the loaded ion exchanger is converted into the final formulation.

10. Use of cation exchangers loaded with active substances, at least 90% of the exchanger particles having a size of less than 50 μm, for producing pharmaceutical preparations for oral administration.
